(19) United States
(12) Patent Application Publication
(10) Pub. No.: US 2018/0169397 A1
(43) Pub. Date: Jun. 21, 2018

(54) CONNECTING DEVICE FOR A CATHETER, IN PARTICULAR A PERIPHERAL VEIN CATHETER

(71) Applicant: SICOS GMBH, Duesseldorf (DE)

(72) Inventors: Kai Markus, Eschweiler (DE); Christian Frauenrath, Juelich (DE); André Reichardt, Apolda (DE)

(21) Appl. No.: 15/309,476

(22) PCT Filed: Apr. 30, 2015
(86) PCT No.: PCT/DE2015/100177

§ 371 Date: Sep. 20, 2017

(30) Foreign Application Priority Data
May 8, 2014 (DE) 10 2014 106 442.9

(51) Int. Cl. A61M 39/16; A61M 5/165

(52) U.S. Cl. CPC A61M 39/162; A61M 2205/7518; A61M 2205/0205; A61M 5/165

(57) ABSTRACT

The invention relates to a connection device for a catheter, especially for a peripheral venous catheter, comprising a main body (1) with a main body infusion channel (2) and a through-flow element (7a, 7b, 7c) having sterilizing effect and extending in the form of a bag in flow direction of the main body infusion channel (2), and is characterized in that an attachment body (10) having an attachment body infusion channel (16) is detachably fixed to the main body (1) and the through-flow element (7a, 7b, 7c) extends at least also into the attachment body infusion channel (16) and is mounted displaceably in the longitudinal direction of the main body infusion channel (2).

The invention relates to a connection device for a catheter, especially for a peripheral venous catheter, with the features of the preamble of claim 1.

A connection device of the type initially mentioned is known from EP 1 579 878 B1, which is particularly suitable for central venous catheters, which remain on the human body for long periods of time, in order to reduce the risk of septa caused by the entry of bacteria. Central venous catheters can be used e.g. for the artificial diet at short-bow syndrome. Such septa are associated with considerable personal disadvantages for the treated person. As a solution, the above-mentioned prior art proposes to provide a sieve element being made of silver, having numerous openings and being arranged in the infusion channel or, seen in the direction of the infusion flow, at the end of the infusion channel. The though-flow element designed as a sieve element represents a barrier for bacteria and/or fungi and is also advantageous for the reason that the complete connection device together with the sieve element can be replaced in a very simple manner, in case the sieve element should become ineffective, e.g. as a result of clogging.

It is an object of the present invention to provide a connection device of the type mentioned at the outset which can further improve the protective effect against sepses.

This object is achieved in a connection device of the type mentioned at the outset by the feature of claim 1. Exemplary and advantageous embodiments of the connection device are defined in the dependent claims.

According to this it is initially proposed to detachably fix an attachment body having an attachment body infusion channel to the main body and to mount the through-flow element displaceably in the longitudinal direction of the main body infusion channel, wherein the though-flow element extends at least also into the attachment body infusion channel.

The through-flow element thus also acts sterilizing on a partial section in the attachment body infusion channel. By means of a long-stretched bag shape the sterilizing effect of the through-flow element can be ensured over a correspondingly long distance. With the detachability of the attachment body on the main body the through-flow element is easily accessible for cleaning or replacement.

The floating mounting of the through-flow element, preferably in the main body, ensures that the distance between the outer wall of the through-flow element and the inner wall of the attachment body infusion channel can vary in dependency on the flow rate given in the infusion channel. At low flow rates, the distance between the through-flow element and the wall of the attachment body infusion channel can remain small. If the attachment body infusion channel also has a sterilizing effect, the ratio of the sterilizing surface to the volume of the liquid in contact with the sterilizing surface is very large which means increased protection. If the flow rate has to be increased, a too small distance between the through-flow element and the infusion channel wall may possibly have an undesirably flow-inhibiting effect. The floating mounting of the through-flow element now allows an automatically resulting distance increase and thus a lower flow resistance.

For this purpose, it may be advantageous that the shape of the wall of the infusion channel surrounding the through-flow element is aligned, at least in part, to the shape of the through-flow element, in particular by a parallel course.

The through-flow element is bag-shaped, whereby a “bag shape” also comprises shapes tapering in flow direction. A tapering may be advantageous, in particular a conical tapering or a tapering in truncated cone shape, seen in the direction of flow. A corresponding shape could be provided by the infusion channel, at least in part, in the area surrounding the through-flow element, preferably with a parallel course of through-flow element outer wall and infusion channel inner wall.

The through-flow element has, preferably, a wall with sieve openings, for example in a circumferential wall and/or in an end wall opposite an inlet opening of the through-flow element, and can, for example, consist of silver or is coated on the surface with silver. Instead of silver a silver-containing alloy or chemical compound or a different sterilizing agent can be used, for example copper or, for example, a plastic with bactericidal, antibiotic and/or cytostatic additives. The additive may, for example, be silver or copper, for example in form of embedding or doping.

The connection device according to the invention can also be configured such that the through-flow element has a liquid-tight circumferential wall and in an end wall opposite of an inlet opening at least one outlet opening, and that the through-flow element undergoes a sealing press fit with the attachment body infusion channel when the attachment body is connected to the main body. In this case the through-flow element is impermeable in the circumferential wall for a liquid, in particular for an infusion fluid, which on these grounds enters the attachment infusion channel only through the end wall. Since the press fit between the through-flow element and the attachment body infusion channel is sealing, the tightness of the connection between the main body and the attachment body with respect to the infusion fluid can be ensured. With the sealing press fit further sealing measures can be supported or become dispensable. Furthermore, with the press fit production tolerances on the dimensions of the attachment body infusion channel can be absorbed to some extent. In this case the displaceable mounting of the through-flow element must also be designed to be liquid tight.

If the mounting of the through-flow element is provided in the main body, the displaceability of the mounting in the longitudinal direction of the infusion channel is advantageous in the case of the press fit between the flow-through element and the inner wall of the connection body infusion channel since in this way tolerances during the manufacture of the attachment body can be absorbed. With the displaceable mounting it is also possible to realize the press fit for different designs or dimensions of the attachment body.

In order to achieve the press fit, the through-flow element and the inner wall of the attachment body infusion channel have suitable shapes. For example, the inner wall of the attachment body infusion channel can taper in the direction of flow of the infusion liquid, for example, the inner wall can be formed conically with a certain opening angle. In this case, the through-flow element may have a cylindrically shaped outer wall or as well taper in the direction of flow, wherein the given per unit length diameter reduction of the outer side of the circumferential wall of the through-flow element is less than the given per unit length diameter reduction of the inner wall of the attachment body infusion channel. In the case of a conical shape, the outer side of the circumferential wall of the through-flow element has a smaller opening angle than the inner wall of the attachment body infusion channel.

A plurality of outlet openings can be provided in the end wall so that a sieve-like structure occurs. Alternatively or additionally, one or more larger outlet opening(s) can be provided in the end wall.

The connection device according to the invention can also be configured in such a way that at least one sterilizing insert body is arranged in the interior of the through-flow element. The at least one an insert body increases the sterilizing surface and can be used independently of the number of outlet openings. The at least one insert body can e.g. have a cruciform cross-section viewed in the direction of flow. The at least one insert body in the interior of the through-flow element can be separate and interchangeable or likewise integral with the through-flow element. The at least one insert body can be made of the same material as the main body. However, the insert body or at least one of the insert bodies can also consist of a material other than that of the main body. As material of the at least one insert body, for example massive silver or copper, or a massive alloy containing silver and/or copper, or a sterilizing doped plastic may be suitable, wherein in case of several insert bodies these may consist of different materials.

To achieve an additional sterilizing effect of the connection device provision can also be made for the main body also to have at least a sterilizing effect in the main body infusion channel, for example by using a sterilizing massive material or a sterilizing coating or doping of the main body. By a massive material is meant a material which has a shape stability without application to a carrier or a supporting substrate. Foils or thin films applied to a substrate are thus not massive material. However, a massive material can be a matrix doped with foreign substances or containing foreign elements, for example reinforcing elements, in particular fibers. The matrix material itself and/or the foreign substances or foreign elements may have a sterilizing effect.

The connection device according to the invention can also be configured in such a way that the attachment body has, at its end facing away from the main body, a coupling piece for connection to a cannula, in particular a butterfly cannula. In this way, the application of a catheter can be speeded up and simplified. Intermediate elements, for example, in the form of a hose, which may also be contaminated and the attachment of which is time consuming, become dispensable therewith.

However, it may also be advantageous to configure the connection device according to the invention in such a way that the attachment body is part of a cannula, in particular a butterfly cannula.

It can also be advantageous to provide a sealing press fit between the main body and the attachment body. This provides an (additional) protection against the leakage of infusion liquid to the outside or the unwanted entry of substances.

In the following, preferred embodiments of the invention are described on the basis of figures.

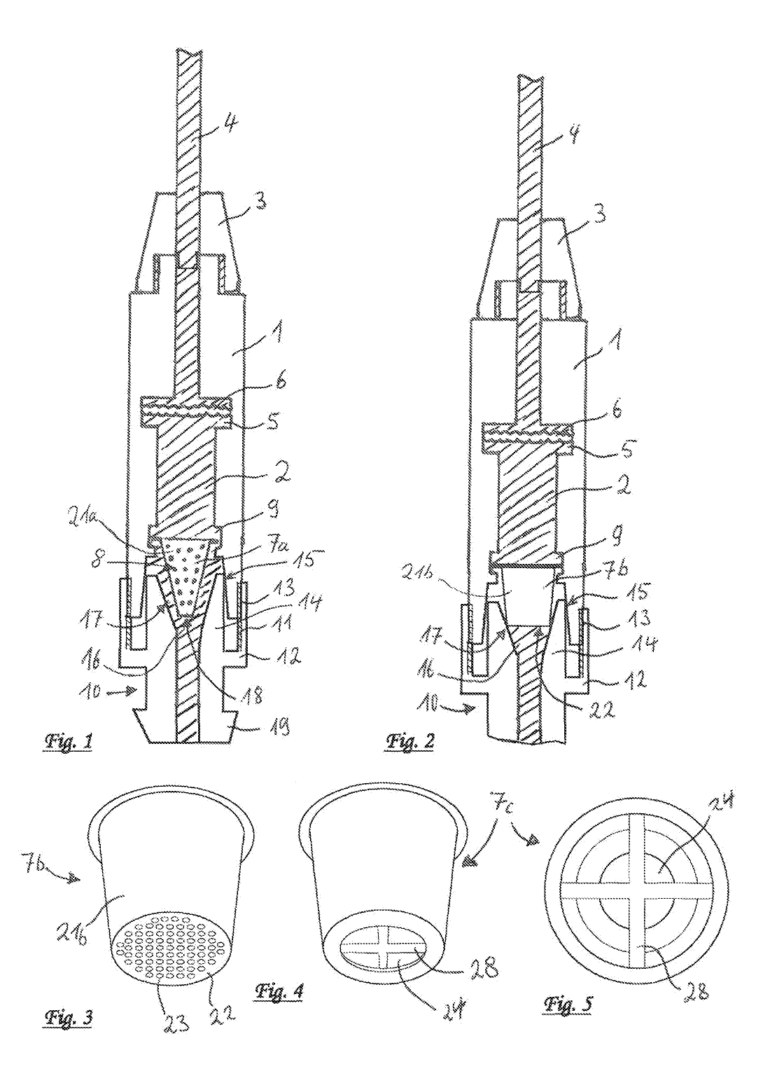

FIG. 1 shows a first embodiment of a connection device with sieve holes in the circumferential wall of a first variant of a through-flow element,

FIG. 2 shows a second embodiment of a connection device with press fit between a second variant of a through-flow element and an infusion channel of a connecting body,

FIG. 3 shows the second variant of the through-flow element with a sieve-like bottom in a perspective view,

FIG. 4 shows a third variant of the through-flow element with sterilizing insert body in a perspective view and

FIG. 5 shows the through-flow element according to FIG. 4 in a top view.

In a schematic representation FIG. 1 shows a first connection device with a main body 1, through which a main body infusion channel 2 runs. The main body 1 is coupled to an infusion tube 4 via a connecting element 3. A widening 5 is provided in the main body infusion channel 2 and has a bacterial filter 6.

At the lower end of the main body infusion channel 2 is a sieve-like through-flow element 7a, which has numerous sieve openings 8 in its circumferential wall 21a. The through-flow element 7a consists of a sterilizing material and/or is coated with a sterilizing material. The sterilizing material may be silver. The through-flow element 7a is mounted so as to float in a recess 9 provided in the main body infusion channel 2 in such a way that the through-flow element 7a can be displaced in the longitudinal direction of the main body 1.

An attachment body 10 is fixed by means of a nut element 12, having an inner thread 11, on an external thread 13 of the main body 1. Attachment body 10 has an engagement fitting 14, which engages in an end region 15 of the main body infusion channel 2. The engagement fitting 14 is made of a flexible material and dimensioned in such a way relative to the end region 15 of the main body 1 that, when fixing the attachment body 10, a press fit results between the engagement fitting 14 and the end region 15 of the main body 1.

With the connection body 10 fixed, the through-flow element 7a projects into an attachment body infusion channel 16. In an input region 17 the attachment body infusion channel 16 is widened, wherein the inner wall of the input region 17 is essentially parallel to the outer wall of the conical-shaped through-flow element 7a.

The through-flow element 7a has, at its end, an end wall 18 also having fine outlet openings, i.e. sieve openings 8, and being oriented perpendicular to the longitudinal direction of the main body infusion channel 2. As shown, the end wall 18 can be flat or have any other, for example also bulging, shape. This ensures that a flow is provided even when the peripheral wall of the flow-through element 7a rests on the inner wall of the attachment body infusion channel 16 in the inlet region 17, which can result in a closure of the screen holes 8 of the end wall 18.

At its end, the attachment body 10 has a coupling piece 19 for connection to a cannula, not shown here, in particular to a butterfly cannula. The coupling piece 19 is merely schematically indicated here and can have a completely different shape and coupling mechanism, e.g. can be part of a Luer lock.

FIG. 2 shows a further variant of a connection device, which in many features corresponds to the connection device according to FIG. 1. Corresponding elements are therefore provided with the same reference numbers. Reference is made here to the description of FIG. 1. In contrast to FIG. 1, a second variant of a through-flow element 7b is provided in the embodiment according to FIG. 2, which is shown enlarged in FIG. 3 in a perspective view obliquely from below. The circumferential wall 21b of the through-flow element 7b is conical, but has a significantly smaller opening angle than the inner wall of the engagement fitting 14. The connection between the main body 1 and the attachment body 10 results in a press fit between the through flow element 7b and the engagement fitting 14. The circumferential wall 21 of the through-flow element 7b has no sieve openings. On the other hand, an end wall 22 has a plurality of sieve openings 23. As far as the mounting of through-flow element 7b in the recess 9 of the main body 1 is configured sealingly in relation to the infusion liquid, the through-flow element 7b has a sealing function as a whole since the press fit between through-flow element 7b and the engagement fitting 14 is also sealing. A sealing function in the connection between intermediate piece 10 and main body 1 is thus no longer required or is at least supported by this embodiment of the through-flow element 7b.

The through-flow element 7b can, of course, also be cylindrically shaped in its peripheral wall 21.

FIGS. 4 and 5 show an alternative embodiment of a through-flow element 7c, which can be used instead of the through-flow element 7a or 7b. Through-flow element 7c has only one single outlet opening 24 instead of sieve openings, wherein a sterilizing cross-shaped insert body 28 is provided in the through-flow element 7c.

### LIST OF REFERENCE NUMBERS

1 Main body

2 Main body infusion channel

3 Connecting element

4 Infusion tube

5 Widening

6 Bacterial filter

7 Through-flow element

8 Sieve opening

9 Recess

10 Attachment body

11 Inner thread

12 Nut element

13 External thread

14 Engagement fitting

15 End region

16 Attachment body infusion channel

17 Input region

18 End wall

19 Coupling piece

20 Through-flow element

21 Circumferential wall

22 End wall

23 Sieve openings

24 Outlet opening

28 Insert body

1-10. (canceled)

11. A connection device for a catheter, especially for a peripheral venous catheter, comprising
a main body with a main body infusion channel and a through-flow element having sterilizing effect and extending in the form of a bag in flow direction of the main body infusion channel, wherein
an attachment body having an attachment body infusion channel is detachably fixed to the main body and
the through-flow element extends at least also into the attachment body infusion channel and is mounted displaceably in the longitudinal direction of the main body infusion channel.

12. The connection device claimed in claim 11 wherein the through-flow element is mounted on the main body (1).

13. The connection device claimed in claim 11 wherein the attachment body infusion channel (16) tapers in the direction of flow at least in a section (17) surrounding the through-flow element (7a, 7b, 7c).

14. The connection device claimed in claim 11 wherein the shape of the wall of the main body infusion channel (2) and/or of the attachment body infusion channel (16), said wall surrounding the through-flow element (7a, 7b, 7c), is aligned, at least in part, to the shape of the through-flow element (7a, 7b, 7c), in particular by a parallel course.

15. The connection device claimed in claim 12 wherein the shape of the wall of the main body infusion channel (2) and/or of the attachment body infusion channel (16), said wall surrounding the through-flow element (7a, 7b, 7c), is aligned, at least in part, to the shape of the through-flow element (7a, 7b, 7c), in particular by a parallel course.

16. The connection device claimed in claim 13 wherein the shape of the wall of the main body infusion channel (2) and/or of the attachment body infusion channel (16), said wall surrounding the through-flow element (7a, 7b, 7c), is aligned, at least in part, to the shape of the through-flow element (7a, 7b, 7c), in particular by a parallel course.

17. The connection device claimed in claim 11 wherein the attachment body (10) has a sterilizing effect in the attachment body infusion channel (16), at least on a section.

18. The connection device claimed in claim 13 wherein the through-flow element (7a, 7b, 7c) has a liquid-tight circumferential wall (21a, 21b, 21c) and in an end wall (18) opposite of an inlet opening at least one outlet opening (23, 24), and that the through-flow element (7a, 7b, 7c) undergoes a sealing press fit with the attachment body infusion channel (16) when the attachment body (8) is fixed to the main body (1).

19. The connection device claimed in claim 11 wherein between main body infusion channel (2) and attachment body infusion channel (16) a sealing press fit is provided.

20. The connection device claimed in claim 11 wherein at least one sterilizing insert body (28) is arranged inside the through-flow element (7a, 7b, 7c).

21. The connection device claimed in claim 14 wherein at least one sterilizing insert body (28) is arranged inside the through-flow element (7a, 7b, 7c).

22. The connection device claimed in claim 15 wherein at least one sterilizing insert body (28) is arranged inside the through-flow element (7a, 7b, 7c).

23. The connection device claimed in claim 16 wherein at least one sterilizing insert body (28) is arranged inside the through-flow element (7a, 7b, 7c).

24. The connection device claimed in claim 11 wherein the attachment body (10) has, at its end facing away from the main body (1), a coupling piece (19) for connection to a cannula, in particular a butterfly cannula.

25. The connection device claimed in claim 11 wherein the attachment body (10) is part of a cannula, in particular of a butterfly cannula.
